(19) United States
(12) Patent Application Publication
(10) Pub. No.: US 2026/0083886 A1
(43) Pub. Date: Mar. 26, 2026

(54) NEGATIVE-PRESSURE ACCESSORY STRUCTURE OF NEGATIVE-PRESSURE INSTRUMENT

(71) Applicants: BIBOTING INTERNATIONAL CO., LTD., TAOYUAN CITY (TW); Po-Chang LIU, TAOYUAN CITY (TW)

(72) Inventor: Po-Chang LIU, TAOYUAN CITY (TW)

(21) Appl. No.: 18/892,305

(22) Filed: Sep. 20, 2024

(51) Int. Cl. A61M 1/08; A61H 9/00

(52) U.S. Cl. CPC A61M 1/08; A61H 9/005; A61H 2201/013

(57) ABSTRACT

A negative-pressure accessory of a negative-pressure instrument is connected to an instrument body through a wire and a suction pipe. The negative-pressure accessory includes a cup body and a pressure-sensing module. The cup body includes a joint connected to one end of the suction pipe. The pressure-sensing module is disposed in the cup body and communicating to the instrument body. When the instrument body sucks an air in the cup body, the pressure-sensing module senses the air flowing therethrough to obtain a negative-pressure signal in the cup body, and the pressure-sensing module transmits the negative-pressure signal to the instrument body to determine a negative-pressure value in the cup body.

### TECHNICAL FIELD

The present disclosure relates to a negative-pressure instrument, particularly relates to a negative-pressure instrument which generates negative-pressure and makes a negative-pressure accessory attach onto a skin of a human body.

### DESCRIPTION OF RELATED ART

As the pace of life becomes faster and faster, modern people often do not have enough time to exercise to relax their body and mind, or a lot of pressure may be accumulated and need to be relieved. Therefore, many kinds of massage and stress relief equipment have appeared on the market. A general negative-pressure instrument mainly includes an instrument body and at least one negative-pressure accessory (cup), the instrument body is connected to a negative-pressure accessory by a suction pipe. The user places the negative-pressure accessory on a part of the body that needs a massage. Under the control of the circuit board inside the instrument body, the negative-pressure is established inside the negative-pressure accessory. As a result, massage and relaxation effects on the human body are achieved.

Additionally, when the instrument body is activated and the user just places the negative-pressure accessory (cup), the negative-pressure accessory (cup) may fall off easily due to the vibration motor on the negative-pressure accessory (cup) starting to vibrate before the negative-pressure accessory (cup) is fully attached onto a part of the skin.

Further, some users are used to turning on the instrument body first, then slowly finding the suitable part of the skin to place the negative-pressure accessory (cup). If the negative-pressure accessory (cup) vibrates first, that may cause a bad experience or make the negative-pressure accessory (cup) be unable to fully attach onto the part of the skin.

Therefore, when the vibrator is activated, how to make the negative-pressure accessory be closely and effectively contact the skin of the human body, and prevent the negative-pressure accessory from falling or slipping is a problem needs to be solved by the present disclosure.

### SUMMARY OF THE DISCLOSURE

Therefore, the main purpose of the present disclosure is to solve the problem of the related-art. The present disclosure provides a negative-pressure accessory, in which a pressure-sensing module is disposed in the cup body of the negative-pressure accessory. During pumping air, the vibrator on the negative-pressure accessory is activated to generate vibration only when the negative-pressure signal inside the negative-pressure accessory is sensed to reach the predetermined pressure. As a result, the negative-pressure accessory may be prevented from slipping or falling during the vibration.

In order to achieve that purpose, the present disclosure provides a negative-pressure accessory of a negative-pressure instrument, connected to an instrument body through a wire and a suction pipe, the negative-pressure accessory including: a cup body and a pressure-sensing module. The cup body includes a joint connected to one end of the suction pipe. The pressure-sensing module is disposed in the cup body and communicatively connected to the instrument body. When the instrument body sucks an air in the cup body, the pressure-sensing module senses the air flowing therethrough to obtain a negative-pressure signal in the cup body, and the pressure-sensing module transmits the negative-pressure signal to the instrument body to determine a negative-pressure value in the cup body.

In some embodiments, the pressure-sensing module includes: an upper cover, a bottom cover, a pressure sensor, and a power supply unit.

In some embodiments, the upper cover includes a joint part in a column shape disposed on a top thereof, the joint part is assembled to an assembly part of the cup body to make the pressure-sensing module be disposed on top of an interior of the cup body, and the cup body includes a vibrator disposed on an outer surface thereof and electrically connected to the instrument body.

In some embodiments, the bottom cover includes a pressure sensing hole defined thereon, and the pressure sensing hole makes the air flow into between the upper cover and the bottom cover for sensing the negative-pressure signal.

In some embodiments, the pressure sensor is fixed to the bottom cover through a plurality of screws, the pressure sensor includes a circuit substrate, a pressure sensing chip, and a communication unit, and the pressure sensing chip and the communication unit are electrically fixed on the circuit substrate.

In some embodiments, the instrument body further includes a base, a case disposed corresponding to a cover of the base, and a panel fixed to a front side of the case, the panel includes a plurality of operation buttons, a knob, a buzzer, a display, a switch, and the other related components, the instrument body further includes a connection interface, a solenoid valve, a circuit board, and a vacuum pump disposed therein, the connection interface is connected to another end of the suction pipe, the solenoid valve is connected to the connection interface through a solenoid valve tube, the vacuum pump is connected to the connection interface through a pump suction tube, and the solenoid valve, the vacuum pump, and the vibrator are electrically connected to the circuit board.

In some embodiments, the circuit board includes: a communication module, communicatively connected to the communication unit of the pressure-sensing module.

In some embodiments, the communication unit and the communication module are a wired module or a wireless module.

In some embodiments, the wired module is a signal cable or a signal transmission line.

In some embodiments, the wireless module is a Bluetooth module or a WIFI module.

### BRIEF DESCRIPTION OF THE DRAWINGS

FIG. 1 is a three-dimensional schematic diagram of the appearance of the instrument body and the negative-pressure accessory of the negative-pressure instrument in accordance with the present disclosure.

FIG. 2 is a schematic diagram of the interior of the instrument body in accordance with the present disclosure.

FIG. 3 is an exploded diagram of the pressure-sensing module in accordance with the present disclosure.

FIG. 4 is a partially cross-sectional diagram of the negative-pressure accessory of FIG. 1 in accordance with the present disclosure.

FIG. 5 is a schematic diagram of using status of the negative-pressure instrument in accordance with the present disclosure.

FIG. 6 is a schematic diagram of the negative-pressure instrument in accordance with another embodiment of the present disclosure.

### DETAILED DESCRIPTION

The technical contents of this disclosure will become apparent with the detailed description of embodiments accompanied with the illustration of related drawings as follows. It is intended that the embodiments and drawings disclosed herein are to be considered illustrative rather than restrictive.

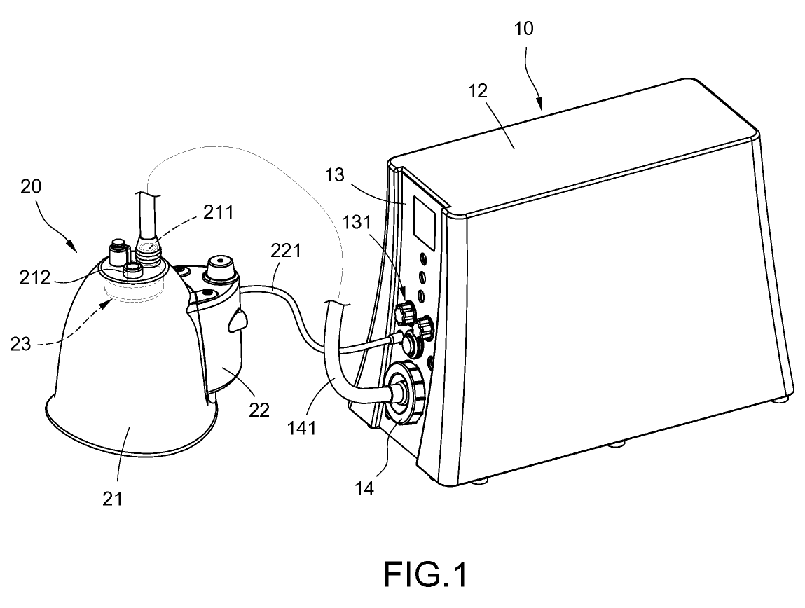

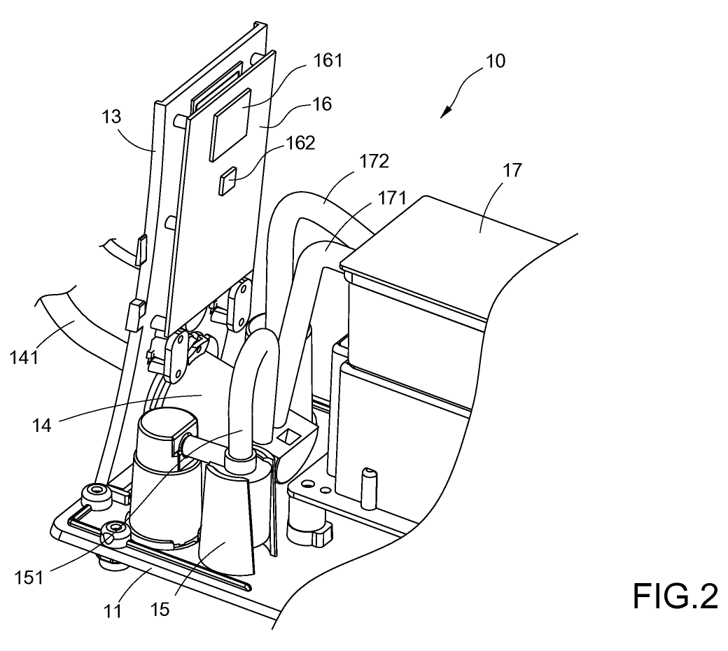

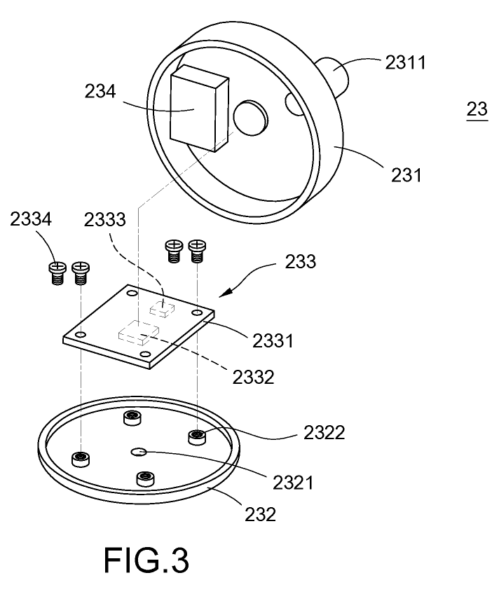

Please refer to FIGS. 1~3, FIG. 1 is a three-dimensional schematic diagram of the appearance of the instrument body and the negative-pressure accessory of the negative-pressure instrument in accordance with the present disclosure, and FIG. 2 is a schematic diagram of the interior of the instrument body in accordance with the present disclosure, and FIG. 3 is a structurally exploded diagram of the pressure-sensing module in accordance with the present disclosure. As shown in the figures, the present disclosure provides a negative-pressure instrument. The negative-pressure instrument includes an instrument body 10 and a negative-pressure accessory 20. The negative-pressure accessory 20 is connected to the instrument body 10 through the power cable 221 and the suction pipe 141, which are collectively assembled to be the negative-pressure instrument.

The instrument body 10 is briefly described. The instrument body 10 includes a base 11, a case 12, and a panel 13. The case 12 is disposed to cover the base 11 correspondingly, and the panel 13 is fixed on the front side of the case 12. The panel 13 includes a plurality of operation buttons 131 and the other related components, for example, a knob, a buzzer, a display, and a switch, etc.

The instrument body 10 includes a connection interface 14, a solenoid valve 15, a circuit board 16, and a vacuum pump 17. The circuit board 16 is used to monitor the pressure value of the negative-pressure inside the negative-pressure accessory 20.

The connection interface 14 is fixed on the panel 13. One end of the connection interface 14 is exposed from the panel 13, and the other end of the connection interface 14 is arranged inside of the instrument body 10. The connection interface 14 has a suction pipe 141 connected to a structure thereof located outside the panel 13. The other end of the suction pipe 141 is connected to the negative-pressure accessory 20.

The solenoid valve 15 is disposed on the base 11 and connected to the connection interface 14 through the solenoid valve tube 151. The solenoid valve 15 is controlled by the circuit board 16 to release the negative-pressure.

The circuit board 16 is disposed on an inner side of the panel 13, and is electrically connected to the operation buttons 131 and the other related components, for example, the knob, the buzzer, the display, the switch, etc., on the panel 13. Moreover, the circuit board 16 is electrically connected to the solenoid valve 15 and the vacuum pump 17. Therefore, by the operation of the operation buttons 131, the circuit board 16 may control the operation of the solenoid valve 15 and the vacuum pump 17.

It is worth mentioning that the circuit board 16 further includes a communication module 162. The communication module 162 receives the negative-pressure signal sensed through the pressure-sensing module 23 through a wired module or a wireless module. The wired module is a signal cable or a signal transmission line. The wireless module is a Bluetooth module or a WIFI module.

The vacuum pump 17 is disposed on the base 11 and is connected, to the connection interface 14 through the pump suction tube 171. The circuit board 16 controls the vacuum pump 17 to make the negative-pressure accessory 20 generate a suction force of negative-pressure. As a result, the negative-pressure accessory 20 may be attached onto a skin of a human body. Besides, the pump releasing tube 172 is connected to the vacuum pump 17 to release gas extracted through the vacuum pump 17 producing the negative-pressure.

The negative-pressure accessory 20 includes a cup body 21, a vibrator 22, and a pressure-sensing module 23. The pressure-sensing module 23 is used to sense the negative-pressure signal in the cup body 21.

The cup body 21 includes a connector 211 disposed thereon, and the connector 211 is connected to the other end of the suction pipe 141. Moreover, the outer top surface of the cup body 21 includes an assembly part 212 which is used to be connected to the pressure-sensing module 23.

The vibrator 22 is disposed on an outer surface of the cup body 21 and is electrically connected to the circuit board 16 through a power cable 221.

The pressure-sensing module 23 includes an upper cover 231, a bottom cover 232, a pressure sensor 233, and a power supply unit 234. The upper cover 231 includes a joint part 2311 in a column shape which is at a top of the upper cover 231. An assembly part 212 on the cup body 21 is connected to the joint part 2311 to make the pressure-sensing module 23 be assembled on top of an interior of the cup body 21. The bottom cover 232 includes a pressure sensing hole 2321 defined thereon. During the air-extraction process of the cup body 21, the pressure sensing hole 2321 makes the air (negative-pressure) flow into between the upper cover 231 and the bottom cover 232. The circuit substrate 2331 of the pressure sensor 233 is screwed and fixed on the threaded hole 2322 of the bottom cover 232 through a plurality of screws 2334. The pressure sensor 233 includes a circuit substrate 2331, a pressure sensing chip 2332, and a communication unit 2333. The pressure sensing chip 2332 and the communication unit 2333 are electrically fixed on the circuit substrate 2331. The pressure sensing chip 2332 is used to sense the negative-pressure signal in the cup body 21. The communication unit 2333 is used to transmit the sensed negative-pressure signal to the communication module of the circuit board 16 to receive. The processor 161 on the circuit board 16 is used to determine whether the negative-pressure value reaches the predetermined negative-pressure value. In this figure, the communication unit 2333 is a wired module or a wireless module. The wired module is a signal cable or a signal transmission line. The wireless module is a Bluetooth module or a WIFI module. Moreover, the power supply unit 234 is disposed between the upper cover 231 and the bottom cover 232 to provide the required power of the pressure sensor 233. In this figure, the power supply unit 234 is a battery or rechargeable battery.

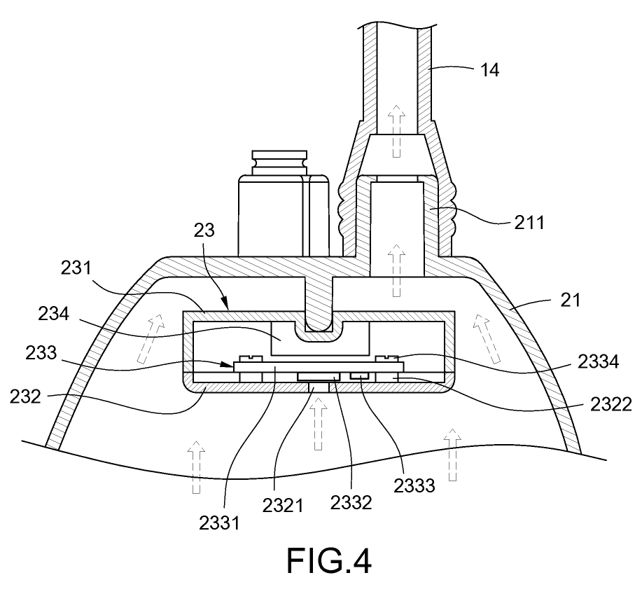

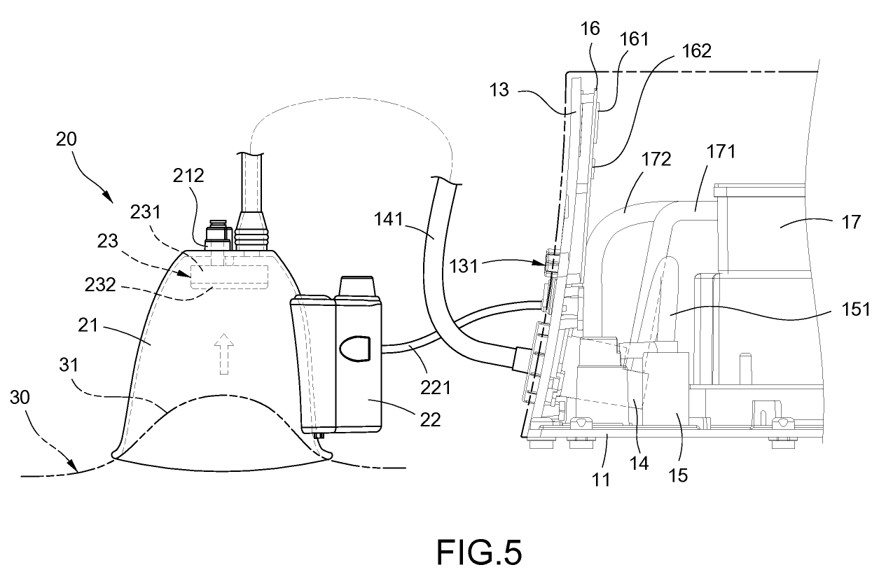

Please refer to FIG. 4 and FIG. 5, FIG. 4 is a partial cross-section diagram of the negative-pressure accessory of FIG. 1 in accordance with the present disclosure and FIG. 5 is a schematic diagram of using status of the negative-pressure instrument in accordance with the present disclosure. As shown in the figure, when the negative-pressure instrument in the present disclosure is in use, the cup body 21 of the negative-pressure accessory 20 is placed on the skin 31 of the human body 30 in advance. At the same time, the operation buttons 131 are used to input signals to the circuit board 16 to make the circuit board 16 drive the vacuum pump 17.

When the vacuum pump 17 is in operation, the air (gas) between the cup body 21 and the skin 31 may be extracted. The extracted air flows through the suction pipe 141, the connection interface 14, and the pump suction tube 171 to the vacuum pump 17, and then is released through the pump releasing tube 172. As a result, the negative-pressure is established between the cup body 21 and the skin 31 and makes the cup body 21 attach onto the skin 31 of the human body 30. In addition, during the air-extraction process, the air (negative-pressure) may flow into the pressure sensing hole 2321 of the bottom cover 232 (as shown in FIG. 4) and be sensed by the pressure sensing chip 2332 of the circuit substrate 2331. Then, the negative-pressure signal sensed by the pressure sensing chip 2332 is transmitted from the communication unit 2333 to the communication module 162 of the circuit board 16 to receive. The processor 161 on the circuit board 16 determines whether the negative-pressure value reaches the predetermined negative-pressure value. If the negative-pressure value reaches the predetermined pressure value, the circuit board 16 controls the vacuum pump 17 to stop operating. Simultaneously, the circuit board 16 immediately drives the vibrator 22 on the cup body 21 to vibrate for massaging and relieving the human body 30.

If the processor 161 of the circuit board 16 determines that the signal sensed through the pressure sensing chip 2332 does not reach the predetermined negative-pressure value, the vacuum pump 17 may continuously operate. In the air suction process, when the negative-pressure value inside the cup body 21 does not reach the predetermined pressure value within a predetermined time, the circuit board 16 may drive the buzzer generating a warning, and the display may display that the cup body 21 is not placed properly and the cup body 21 is not in full gas-tight contact with the skin 31. As a result, the user may replace the cup body 21 to be firmly in contact with the skin 31 surfaces of the human body 30.

After the treatment, the signal is inputted to the circuit board 16 through the operation buttons 131, the circuit board 16 may drive the solenoid valve 15 to release the gas. After the negative-pressure inside the cup body 21 is released, the attachment between the cup body 21 and the skin 31 of the human body 30 through the negative-pressure is released, and the cup body 21 may be removed.

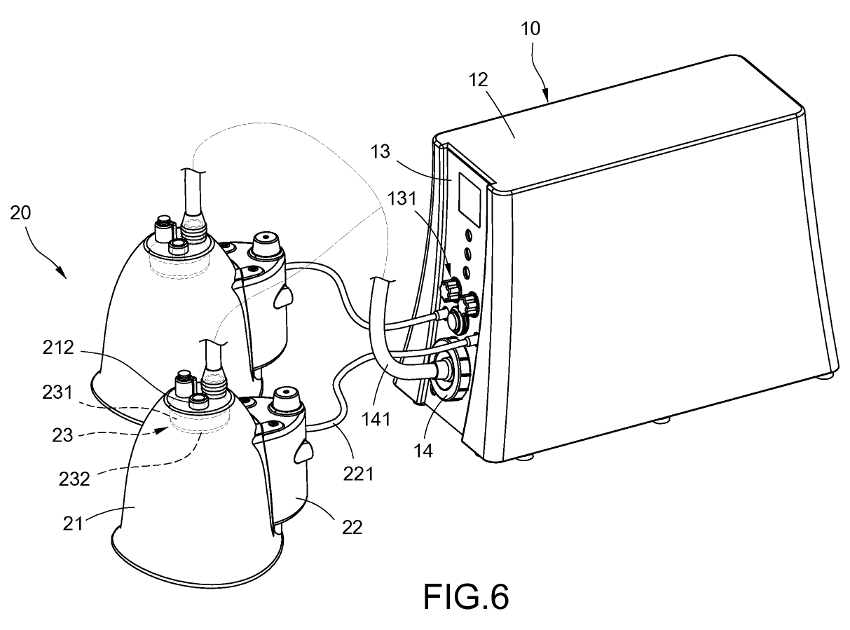

Please refer to FIG. 6, FIG. 6 is a schematic diagram of the negative-pressure instrument in accordance with another embodiment of the present disclosure. As shown in the figure, this embodiment is similar to FIGS. 1 to 5. The difference is that a divergent connector (not shown in the figures) may be connected to a plurality of negative-pressure accessories 20 through the suction pipe 141. The controlling manners of one or more of the negative-pressure accessories 20 are the same. When the negative-pressure is established between each cup body 21 and the skin 31, the negative-pressure value inside each cup body 21 is the same. Therefore, when the negative-pressure value inside any one of the cup bodies 21 does not reach the predetermined pressure value within a predetermined time, the circuit board 16 may drive the buzzer generating a warning, and the display may display that the cup body 21 is not placed properly and the cup body 21 is not in full gas-tight contact with the skin 31. As a result, the user may replace the cup body 21 to be firmly in contact with the skin 31 surfaces of the human body 30.

While this disclosure has been described by means of specific embodiments, numerous modifications, and variations may be made thereto by those skilled in the art without departing from the scope and spirit of this disclosure set forth in the claims.

What is claimed is:

1. A negative-pressure accessory of a negative-pressure instrument, connected to an instrument body through a wire and a suction pipe, the negative-pressure accessory comprising:
a cup body, comprising a joint connected to one end of the suction pipe; and
a pressure-sensing module, disposed in the cup body and communicatively connected to the instrument body;
wherein, when the instrument body sucks an air in the cup body, the pressure-sensing module senses the air flowing therethrough to obtain a negative-pressure signal in the cup body, and the pressure-sensing module transmits the negative-pressure signal to the instrument body to determine a negative-pressure value in the cup body.

2. The negative-pressure accessory of claim 1, wherein, the pressure-sensing module comprises:
an upper cover, a bottom cover, a pressure sensor, and a power supply unit.

3. The negative-pressure accessory of claim 2, wherein, the upper cover comprises a joint part in a column shape disposed on a top thereof, the joint part is assembled to an assembly part of the cup body to make the pressure-sensing module be disposed on top of an interior of the cup body, and the cup body comprises a vibrator disposed on an outer surface thereof and electrically connected to the instrument body.

4. The negative-pressure accessory of claim 2, wherein, the bottom cover comprises a pressure sensing hole defined thereon, and the pressure sensing hole makes the air flow into between the upper cover and the bottom cover for sensing the negative-pressure signal.

5. The negative-pressure accessory of claim 3, wherein, the pressure sensor is fixed to the bottom cover through a plurality of screws, the pressure sensor comprises a circuit substrate, a pressure sensing chip, and a communication unit, and the pressure sensing chip and the communication unit are electrically fixed on the circuit substrate.

6. The negative-pressure accessory of claim 5, wherein, the instrument body further comprises a base, a case disposed corresponding to a cover of the base, and a panel fixed to a front side of the case, the panel comprises a plurality of operation buttons, a knob, a buzzer, a display, and a switch, the instrument body further comprises a connection interface, a solenoid valve, a circuit board, and a vacuum pump disposed therein, the connection interface is connected to another end of the suction pipe, the solenoid valve is connected to the connection interface through a solenoid valve tube, the vacuum pump is connected to the connection interface through a pump suction tube, and the solenoid valve, the vacuum pump, and the vibrator are electrically connected to the circuit board.

7. The negative-pressure accessory of claim 6, wherein, the circuit board comprises:
a communication module, communicatively connected to the communication unit of the pressure-sensing module.

8. The negative-pressure accessory of claim 7, wherein, the communication unit and the communication module are a wired module or a wireless module.

9. The negative-pressure accessory of claim 8, wherein, the wired module is a signal cable or a signal transmission line.

10. The negative-pressure accessory of claim 8, wherein, the wireless module is a Bluetooth module or a WIFI module.
